(19) United States
(12) Patent Application Publication
(10) Pub. No.: US 2015/0361139 A1
(43) Pub. Date: Dec. 17, 2015

(54) CRYSTALLINE FORM OF LINACLOTIDE

(71) Applicant: SANDOZ AG, Basel (CH)

(72) Inventors: Marijan STEFINOVIC, Kundl (AT); Myriam SCANSETTI, Edinburgh (GB); Hayley REECE, Edinburgh (GB)

(21) Appl. No.: 14/761,852

(22) PCT Filed: Jan. 28, 2014
(86) PCT No.: PCT/EP2014/051636

§ 371 Date: Jul. 17, 2015

(30) Foreign Application Priority Data
Jan. 30, 2013 (EP) 13153224.4
Nov. 29, 2013 (EP) 13195100.6

(51) Int. Cl. C07K 7/08

(52) U.S. Cl. CPC C07K 7/08; A61K 38/00

(57) ABSTRACT

Linaclotide is a guanylate cyclase type C receptor (GCC) agonist used in the treatment of gastrointestinal disorders and conditions, including irritable bowel syndrome and chronic constipation. Crystalline form II of Linaclotide is prepared in high purity and yields and shows superior chemical stability in comparison to known crystal or amorphous forms of Linaclotide. A process for the purification of Linaclotide is also provided.

### FIELD OF INDUSTRIAL APPLICABILITY

The present invention relates to a polymorphic form of Linaclotide, processes for its preparation, compositions comprising it and their medical use. It also relates to processes for preparing amorphous Linaclotide making use of said crystalline form.

### BACKGROUND OF THE DISCLOSURE

Linaclotide is a guanylate cyclase type C receptor (GCC) agonist that stimulates the production of cyclic guanosine monophosphate (cGMP). Linaclotide is a 14-amino-acid cyclic peptide with three disulfide bonds, the sequence consisting of cyclized (Cys-Cys-Glu-Tyr-Cys-Cys-Asn-Pro-Ala-Cys-Thr-Gly-Cys-Tyr with disulfide bridges between the cysteine residues at positions 1 and 6, 2 and 10, and 5 and 13. Its preparation by solid phase synthesis is disclosed in WO 2004/069165 A2 and in Current Opinion in Molecular Therapeutics 2007, 9(4), 403-410. Linaclotide may be administered orally for the treatment of gastrointestinal disorders and conditions, including irritable bowel syndrome and chronic constipation. Solid formulations comprising Linaclotide have been developed for oral administration.

WO2010/115916 A1 describes methods of isolation of amorphous Linaclotide from hydroalcoholic of heptane solutions, or by spray drying. Such isolation processes are lengthy and require repeated stripping with different solvents. The resulting product has a lower purity than the starting material due to its “baking” during solvent evaporation.

WO 2010/059733 A1 discloses a crystalline form of Linaclotide designated as form alpha. It also discloses that amorphous Linaclotide is obtained following the procedure described in WO 2004/069165 A2. Form alpha is prepared using aqueous acid and amorphous Linaclotide and is claimed to have a greater chemical stability than the amorphous material. Acid-induced degradation products are however formed to a larger extent during storage of form alpha in comparison to the amorphous form.

An object of the present invention is thus the provision of a crystalline form of Linaclotide having increased chemical stability.

Purification of peptides is usually effected by column chromatography, a procedure that is little suitable to an industrial scale. A further object is thus the provision of a process for the purification of Linaclotide in a simple and efficient way.

### SUMMARY OF THE DISCLOSURE

The present disclosure provides a crystalline form of Linaclotide, which has been designated crystal form II, and a process for its preparation. It also provides a process for the purification of Linaclotide by crystallization and isolation of Linaclotide crystal form II. Crystal form II can be obtained in high chemical purity, possesses superior chemical stability and it can be used in the manufacture of substantially pure amorphous Linaclotide.

### BRIEF DESCRIPTION OF THE DRAWINGS

FIG. 1 illustrates the x-ray powder diffraction patterns of crystalline form II of Linaclotide.

FIG. 2 illustrates the comparison between the x-ray powder diffraction patterns of crystalline form II of Linaclotide and amorphous Linaclotide obtained after washing of the former with heptane.

FIG. 3 illustrates the appearance under polarized light microscopy of crystalline form II of Linaclotide.

FIG. 4 illustrates the comparison between the stability of crystal form alpha and crystal form II.

FIG. 5 illustrates the DSC trace of crystalline form II.

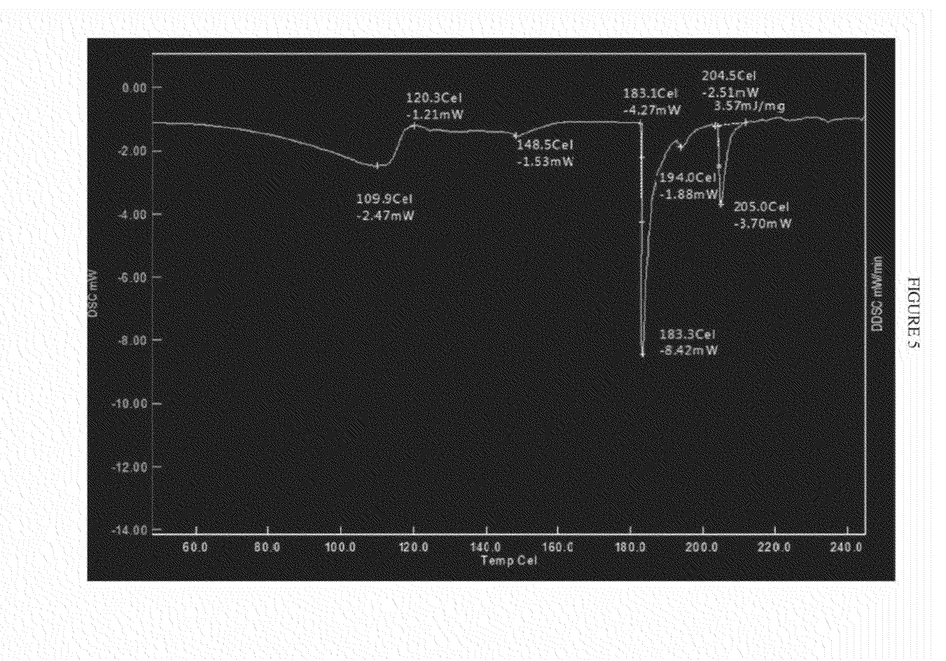

FIG. 6 illustrates crystal packing overlay of Linaclotide molecules obtained from 1,2-propane diol with Linaclotide molecules obtained from ethylene glycol.

FIG. 7 illustrates the crystal packing overlay of Linaclotide molecules obtained from 1,3-propane diol with Linaclotide molecules obtained from ethylene glycol.

### DETAILED DESCRIPTION OF THE DISCLOSURE

The disclosure relates to a crystalline form of Linaclotide, which is described and characterized herein.

### Definitions

As used herein “polymorph” refers to crystalline forms having the same chemical composition but different spatial arrangements of the molecules, atoms, and/or ions forming the crystal.

As used herein “hydrate” refers to a crystal line form of a molecule that further comprises molecules of water incorporated into the crystalline lattice structure. The water molecules in the hydrate may be present in a regular arrangement and/or a non-ordered arrangement. The hydrate may comprise either a stoichiometric or nonstoichiometric amount of the water molecules. For example, a hydrate with a nonstoichiometric amount of water molecules may result from partial loss of water from the stoichiometric hydrate. Hydrate may occur as dimers or oligomers comprising more than one molecule or Linaclotide within the crystalline lattice structure.

As used herein “solvate” refers to a crystalline form of a molecule that further comprises molecules of solvent incorporated into the crystalline lattice structure. The solvent molecules in the solvate may be present in a regular arrangement and/or a non-ordered arrangement. The solvate may comprise either a stoichiometric or nonstoichiometric amount of the solvent molecules. For example, a solvate with a nonstoichiometric amount of solvent molecules may result from partial loss of solvent from the stoichiometric solvate. Solvates may occur as dimers or oligomers comprising more than one molecule or Linaclotide within the crystalline lattice structure.

As used herein, “isostructural solvate” refers to a compound crystalline lattice having a plurality of repeating cavities wherein some or all of the cavities may optionally be occupied by solvent molecules which are the same or different.

As used herein “amorphous” refers to a solid form of a molecule that is not crystalline. As amorphous solid does not display a definite X-ray diffraction pattern.

As used herein, the term “substantially pure” with reference to a particular polymorphic form means that the polymorphic form includes less than 10%, preferably less than 5%, more preferably less than 3%, most preferably less than 1% by weight of any other physical forms of the compound.

### Crystal Form II

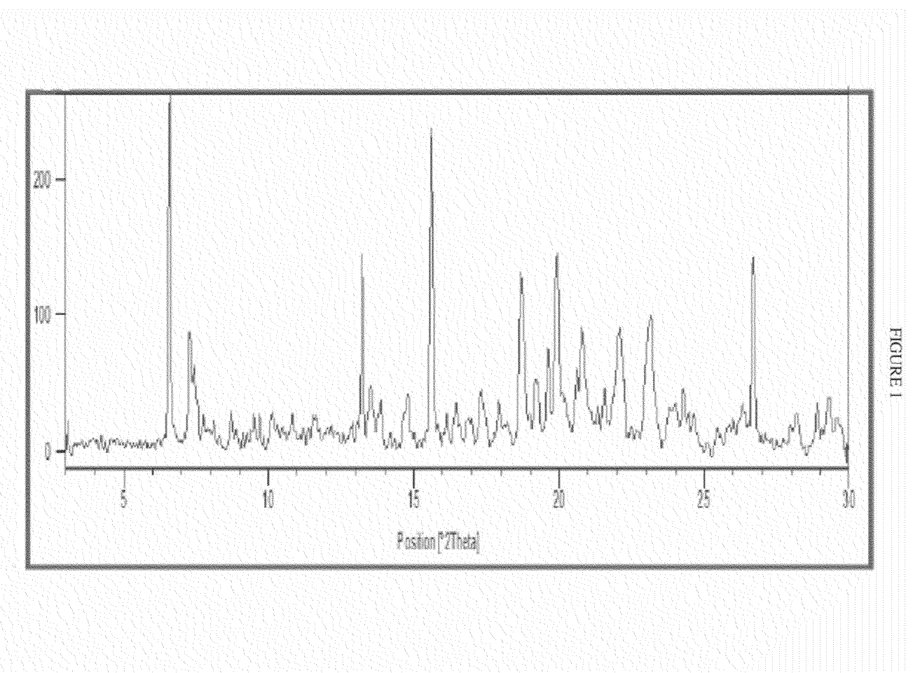

The present invention provides a crystalline form of Linaclotide having an X-ray diffraction pattern substantially the same as the X-ray powder diffraction pattern shown in FIG. 1. The term “essentially the same” with reference to X-ray diffraction peak positions means that typical peak position and intensity variability are taken into account. For example, one skilled in the art will appreciate that the peak positions (2θ) will show some inter-apparatus variability, typically as much as 0.2°. Further, one skilled in the art will appreciate that relative peak intensities will show inter-apparatus variability as well as variability due to degree of crystallinity, preferred orientation, prepared sample surface, and other factors known to those skilled in the art, and should be taken as qualitative measure only. Consequently, it is to be understood that the crystal form of the present invention is not limited to the crystal form that provides X-ray diffraction patterns completely identical to the X-ray diffraction patterns depicted in the accompanying figures disclosed herein. Any crystal forms that provide X-ray diffraction patterns substantially identical to those disclosed in the accompanying Figures fall within the scope of the present invention. The ability to ascertain substantial identities of X-ray diffraction patterns is within the purview of one of ordinary skill in the art. In one embodiment of the present invention, crystalline form II of Linaclotide is provided in substantially pure form.

In one aspect, the invention features a Linaclotide solvate, preferably an isostructural solvate, referred to as “crystal form II”. Crystalline Form II, as disclosed herein, comprises a crystalline lattice of Linaclotide in which voids in the crystalline lattice are empty, or occupied, or partially occupied by one or more molecules of a suitable solvent. Suitable solvents are selected from the group consisting of water, diols, polar aprotic solvents and mixtures thereof, preferably from the group consisting of water, ethylene glycol, 1,2-propanediol, 1,3-propanediol, dimethyl sulfoxide (DMSO), N-methylpyrrolidone (NMP), dimethyl formamide (DMF), dimethyl acetamide (DMA) and mixtures thereof, more preferably fro the group consisting of water, ethylene glycol, 1,2-propanediol, 1,3-propanediol and mixtures thereof. Most preferably the solvent is a mixture of water and any of the above mentioned solvents. Crystalline form II of Linaclotide may contain 0% to 20% of water, preferably 5% to 15% of water, more preferably 10% to 15% of water. It may also contain solvents other than water used in its preparation, preferably from 0% to 30%. Certain physical characteristics of Crystal form II isostructural solvate forms, such as X-ray powder diffraction, melting point, and DSC, are not substantially affected by the particular solvent molecule in question.

Crystalline form II has as orthorhombic P2₁2₁2₁ space group symmetry and the following unit cell dimensions: a=17.1+/−0.5 Å, b=21.8+/−0.7 Å, c=26.4+/−0.8 Å, α=90°, β90°, γ=90° at −153° C.

In a preferred embodiment, crystalline form II obtained from ethylene glycol:water (96:4 v/v) has an orthorhombic P2₁2₁2₁ space group with the following unit cell dimensions: a=17.1+/−0.5 Å, b=21.8+/−0.7 Å, c=26.4+/−0.8 Å, α=90°, β=90°, γ=90° at −153° C.

In a preferred embodiment, crystalline form II obtained from 1,3-propanediol has an orthorhombic P2₁2₁2₁ space group with the following unit cell dimensions: a=17.1+/−0.5 Å, b=22.0+/−0.7 Å, c=26.1+/−0.8 Å, α=90°, β=90°, γ=90° at 20° C.

In a preferred embodiment, crystalline form II obtained from 1,2-propanediol has an orthorhombic P2₁2₁2₁ space group with the following unit cell dimensions: a=17.0+/−0.5 Å, b=22.1+/−0.7 Å, c=26.5+/−0.8 Å, α=90°, β=90°, γ=90° at 20° C.

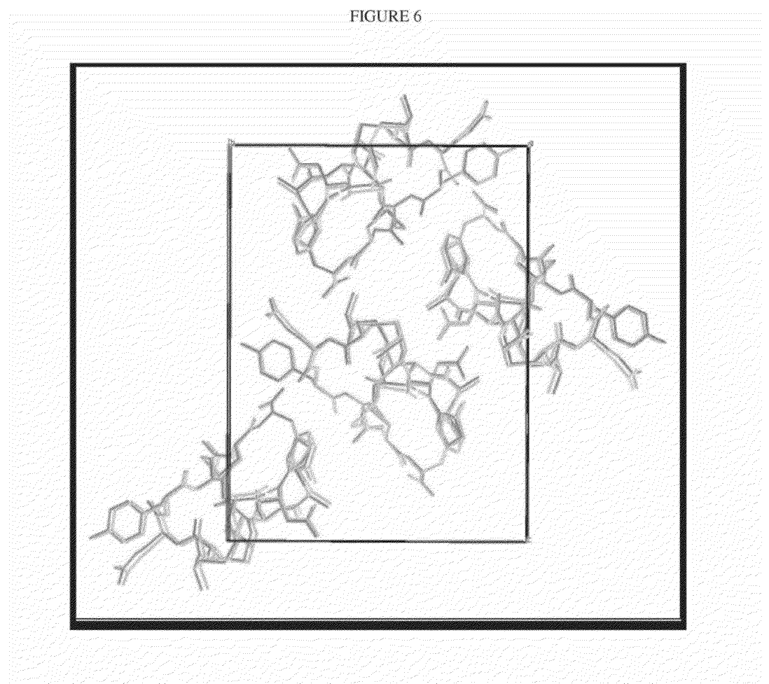

An overlay of the crystal packing of Linaclotide molecules obtained from 1,2-propane diol with that present in crystalline form II obtained from ethylene glycol is shown in FIG. 6.

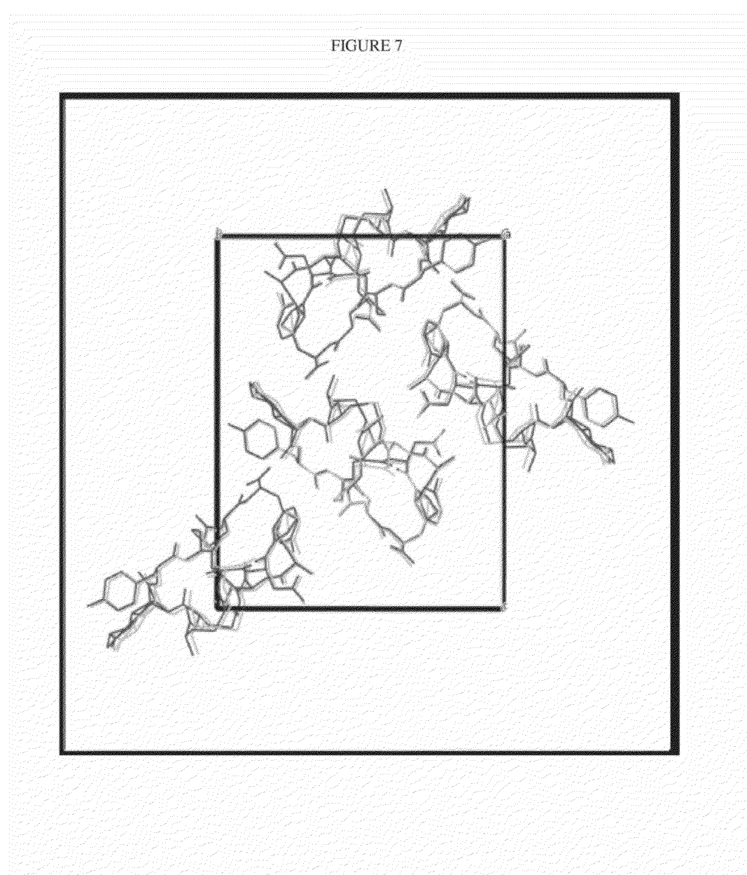

A conformational overlay of crystalline form II obtained from 1,3-propane diol with crystalline form II obtained from ethylene glycol is shown in FIG. 7. Crystalline form II may be characterized by a X-ray powder diffraction pattern (XRPD) comprising peaks at 2θ values of 6.6°, 15.6°, 18.7°, 19.9° and 23.1°, measured at a temperature of about 20° C. and using Cu-Kα radiation (wavelength λ=1.5418 Å). Preferably, crystalline form II may be characterized by a x-ray powder diffraction pattern comprising 2θ values of 6.6°, 7.3°, 8.1°, 15.6°, 18.7°, 19.9°, 23.1° and 26.7°, measured at a temperature of about 20° C. and using Cu-Kα radiation (wavelength λ=1.5418 Å) The complete listing of peaks, their heights and relative intensities are reported in the following table:

| | Height | |
|---|---|---|
| Position 2θ [°] | [cts] | Relative intensity |
| 6.6 | 261 | 100 |
| 7.3 | 94 | 36 |
| 7.8 | 16 | 6 |
| 8.1 | 26 | 10 |
| 8.7 | 24 | 9 |
| 9.5 | 19 | 7 |
| 9.6 | 11 | 4 |
| 9.7 | 19 | 7 |
| 10.1 | 26 | 10 |
| 10.8 | 22 | 8 |
| 11.6 | 22 | 8 |
| 13.2 | 142 | 54 |
| 13.5 | 44 | 17 |
| 13.9 | 32 | 12 |
| 14.8 | 39 | 15 |
| 15.6 | 239 | 91 |
| 16.1 | 24 | 9 |
| 16.5 | 33 | 13 |
| 17.0 | 19 | 7 |
| 17.3 | 41 | 16 |
| 17.9 | 25 | 10 |
| 18.7 | 119 | 46 |
| 19.2 | 48 | 18 |
| 19.6 | 73 | 28 |
| 19.9 | 142 | 54 |
| 20.6 | 77 | 29 |
| 20.8 | 87 | 33 |
| 21.3 | 39 | 15 |
| 21.6 | 57 | 22 |
| 22.1 | 87 | 33 |
| 23.1 | 127 | 49 |
| 23.9 | 27 | 10 |
| 24.3 | 50 | 19 |
| 24.6 | 33 | 13 |
| 25.4 | 14 | 5 |
| 25.9 | 16 | 6 |
| 26.3 | 39 | 15 |
| 26.7 | 139 | 53 |
| 28.2 | 25 | 9 |
| 28.9 | 32 | 12 |
| 29.2 | 35 | 13 |
| 29.6 | 29 | 11 |

In a preferred embodiment, crystal form II is in substantially pure form. Preferably, Form II includes less than 10%, more preferably less than 5%, even more preferably less than 3%, most preferably less than 1% by weight of crystal form alpha.

Crystalline form alpha may be characterized by a X-ray powder diffraction pattern (XRPD) comprising peaks at 2θ values of 6.1°, 8.5°, 11.3°, 12.2° and 22.8°, measured at a temperature of about 20° C. and using Cu-Kα radiation (wavelength λ=1.5418 Å). Preferably, crystalline form II may be characterised by a X-ray powder diffraction pattern comprising 2θ values of 6.1°, 8.5°, 11.3°, 11.8°, 12.2°, 14.3° and 22.8°, measured at a temperature of about 20° C. and using Cu-Kα radiation (wavelength λ=1.5418 Å).

Crystalline form II may be characterized by a DSC trace showing a broad endotherm with onset at about 60° C. followed by two melting endotherms at 183° C. and 205° C.

### Preparation Process

In one method to prepare crystals, Linaclotide or Linaclotide acetate is suspended and/or stirred in a suitable solvent to obtain a slurry, which may be heated to promote dissolution, and then isolating crystalline Linaclotide form II. The term “slurry”, as used herein, means a saturated solution of the compound, which may also contain an additional amount of the compound to afford a heterogeneous mixture of the compound and a solvent at a given temperature.

Suitable solvents for the preparation of crystalline form II are selected from the group consisting of water, diols, polar aprotic solvents and mixtures thereof, preferably from the group consisting of water, ethylene glycol, 1,2-propanediol, 1,3-propanediol, dimethyl sulfoxide (DMSO), N-methylpyrrolidone (NMP), dimethyl formamide (DMF), dimethyl acetamide (DMA) and mixtures thereof, more preferably from the group consisting of water, ethylene glycol, 1,2-propanediol, 1,3-propanediol and mixtures thereof. Most preferably the solvent is a mixture of water and any of the above mentioned solvents. The water content in the solvent is preferably below 20%, more preferably below 10%, even more preferably between 5% and 10% by weight. Diols and polar aprotic solvent are hygroscopic and thus commercial grade solvents contain small amounts water. Typical water content of commercial grades of 1,2-propanediol is from 0.05 to 0.2 weight % and of DMSO from 0.03 to 2 weight %.

Seed crystals may be added to any crystallization mixture to promote crystallization. Seeding may be employed to control growth of a particular polymorph or to control the particle size distribution of the crystalline product. The crystallization mixture may be gently centrifuged or carefully filtered under vacuum to afford the desired crystalline form II. Crystalline form II may be prepared directly from the reaction medium of the final process for preparing Linaclotide. This may be achieved, for example, by employing in the final process stop a solvent or a mixture of solvents from which Linaclotide form II may be crystallized.

Crystalline form II of Linaclotide can be preferably generated using one of the following methods.

### Method A

A slurry is generated by suspending Linaclotide acetate in the suitable solvent identified above. The slurry is then subjected to a temperature cycling regimen where each cycle lasts 1 min to 5 h, preferably 3 h to 4 h. During each cycle the temperature starts at a first value, selected between 0° C. and 10° C. increases over time to a second value, selected between 17 and 27° C., and then drops back down to the first value. Each such cycle is repeated 1 to 25 times, preferably 3 to 10 times. The resulting slurry is then filtered and the isolated solid gently centrifuged to provide the crystalline form of the present invention.

### Method B

Alternatively Form II is dissolved in the suitable solvent identified above and the solvent is slowly evaporated over several hours to several days at a temperature of 15° C. to 30° C.

### Amorphisation

Form II of Linaclotide converts to amorphous Linaclotide on very mild grinding, i.e. under mild pressure, e.g. by grinding in a ball mill for a few minutes at several hours; e.g. 20 min to 5 h depending on the batch size and forces involved. Depending on the time and intensity of milling substantially pure amorphous Linaclotide of the invention is obtained.

Amorphisation can also be achieved by washing form II with a solvent. Suitable solvents for amorphisation are alcohols, such as ethanol or isopropanol, ketones, such as acetone or methyl ethyl ketone, ethers, such as diethyl ether, diisopropyl ether t-butyl methyl ether, hydrocarbons, such as hexane, heptane and toluene, and water. Amorphisation can also be achieved by drying in air or under vacuum.

During the amorphisation process the content of crystalline form II decreases whereas the amount of amorphous Linaclotide increases. The amount of crystalline form II can easily be monitored by XRPD analysis of the mixture.

### Advantages

This method provides a way to access highly pure Linaclotide starting from commercially available lower purity Linaclotide or Linaclotide acetate by rising crystalline form II as an intermediate. Slurrying with solvent obviates the need for expensive chromatographic separation techniques and is far less complex when implemented on larger scales, e.g. kilogram scale. The procedure is carried out under near neutral pH conditions and near ambient temperature, thereby avoiding degradation products resulting from thermal or acid mediated decomposition. Moreover crystalline form II can he converted into amorphous Linaclotide in a simple and efficient way.

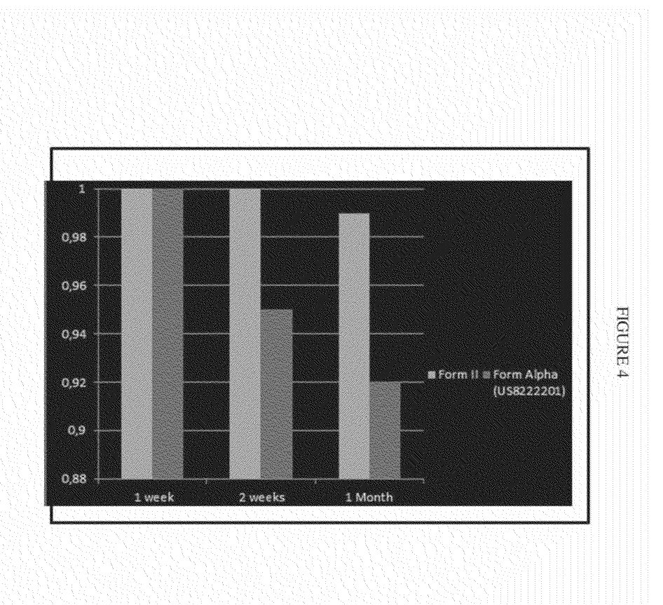

In addition crystalline form II of Linaclotide provides several benefits over the known crystalline form alpha, since it is less susceptible to mechanical manipulation (e.g. centrifugation), losing its crystalline structure and converting to an amorphous state only when washed with solvent or when ground with a mortar and pestle. The very mild conditions needed for the conversion from a crystalline state to an amorphous state preserves the chemical purity of the active pharmaceutical ingredient, e.g. no epimerization nor formation of degradation products is observed during this operation. The superior chemical stability of crystalline form II in comparison with crystalline form alpha known in the art is shown in FIG. 4 and in the table below.

ENVIRONMENT

| | 40° C./75% RH | 40° C./75% RH |
|---|---|---|
| Time | Form II¹ | Form Alpha^{1,2} |
| Day 7 | 100% | 100% |
| Day 14 | 100% | 94.8% |
| Month 1 | 98.9% | 91.9% |

¹Normalised purity
²According to U.S. Pat. No. 8,222,201 (WO 2010/059733).

Form II is less susceptible to degradation than form alpha even in an open atmosphere of 75% RH and 40° C.

### Medical Use and Formulations

The crystalline form of the invention may be used in the treatment of gastrointestinal disorders and conditions, including irritable bowel syndrome and chronic constipation. It may be formulated with one or more excipients or other active pharmaceutical ingredients to provide formulations suitable for the treatment of the indications identified above. Such formulations may optionally include one or more other components selected, for example, from the group consisting of excipients, such as diluents, binders, disintegrants, lubricants, preservatives and coating materials, and other active pharmaceutical ingredients of different molecular structure. Alternatively crystalline form II may be converted to amorphous material as described above, which can be then used for the preparation of suitable finished dosage forms.

### X-ray Powder Diffraction (XRPD)

XRPD analysis was carried out on a Siemens D5000, scanning the samples between 3 and 30°2θ. For small sample amounts, the material was gently compressed onto a glass slide, fitted into an XRPD sample holder.

| | |
|---|---|
| Raw Data Origin | Siemens-binary V2(.RAW) |
| Start Position [°2Th.] | 3.0000 |
| End Position [°2Th.] | 30.000 |
| Step Size [°2Th.] | 0.0200 |
| *Scan Step Time [s] | 1 |
| Scan Type | Continuous |
| Offset [°2Th.] | 0.0000 |
| Divergence Slit Type | Fixed |
| Divergence Slit Size [°] | 2.0000 |
| Specimen Length [mm] | various |
| Receiving Slit Size [mm] | 0.2000 |
| Measurement Temperature [° C.] | 20.00 |
| Anode Material | Cu |
| K-Alpha1 [Å] | 1.54060 |
| K-Alpha2 [Å] | 1.54443 |
| K-Beta [Å] | 1.39225 |
| K-A2/K-A1 Ratio | 0.50000 (nominal) |
| Generator Settings | 40 mA, 40 kV |
| Diffractometer Type | d5000 |
| Goniometer Radius [mm] | 217.50 |
| Incident Beam Monochromator | No |
| Diffracted Beam Monochromator | (Graphite) |
| Spinning | No |

*Note:
For some experiments, in order to improve the signal to noise ratio, the scan step time was increased to 5 or 12 seconds and/or a zero background slide was employed

### Polarised Light Microscopy (PLM)

The presence of birefringence was determined using an Olympus BX50 polarising microscope, equipped with a Motic camera and image capture software. All images were recorded using the 20× objective for routine analysis.

### Single Crystal X-ray Diffraction

Single crystal X-ray diffraction was carried out on an Agilent Supernova dual-source diffractometer equipped with an Oxford Cryosystems low-temperature device operating at 120 K. Data was collected using Cu-Kα radiation (λ=1.54184 Å) to a resolution of 0.9 Å.

### High Performance Liquid Chromatography-Ultraviolet Detection (HPLC-UV)

The purity of solid samples was measured in area percentage by HPLC analysis using the method reported below:

Sample preparation 2 mg/ml, prepared in mobile phase A

Instrument: Agilent 1100

Column: YMCPro, C18, 150×3 mm, 3 μm

Column temperature: 40° C.

λ: 220 nm

Injection volume: 10 μl

Flow rate: 0.6 ml/min

Mobile phase A: 0.1% trifluoroacetic acid in 98:2 H₂O:acetonitrile

Mobile phase B: 0.1% trifluoroacetic acid in 95:5 acetontrile:H₂O

Sample concentration: 2 mg/ml

Gradient program:

| Time (minutes) | Mobile phase B [%] |
|---|---|
| 0.0 | 0 |
| 4 | 0 |
| 9 | 10 |
| 43 | 23 |
| 49 | 34 |
| 59 | 80 |
| 60 | 0 |
| 67 | 0 |

Run time: 25 minutes

### Differential Scanning Calorimetry (DSC)

Approximately, 5 mg of material was weighed into an aluminium DSC pan and sealed non-hermetically with a pierced aluminium lid. The sample pan was then loaded into a Seiko DSC6200 (equipped with a cooler) cooled and held at 25° C. Once a stable heat-flow response was obtained, the sample and reference were heated to 26° C. at scan rate of 10° C./min and the resulting heat flow response monitored.

### Stability Tests

Approximately 10 mg of material was placed in each vial (ea. 1.5 mL, open, clear glass vial).

Three vials were placed into a beaker which was then stored in a sealed desiccator containing a sodium chloride solution which keeps a constant 75% RH. The desiccator was placed into an oven at 40° C.

One vial at a time was taken out after 7 days, 14 days and 1 month and analysed.

Ovens were temperature monitored using calibrated thermometers with min and max temperatures recorded throughout the stability study.

The following non-limiting examples are illustrative of the disclosure.

### EXAMPLES

### Example 1

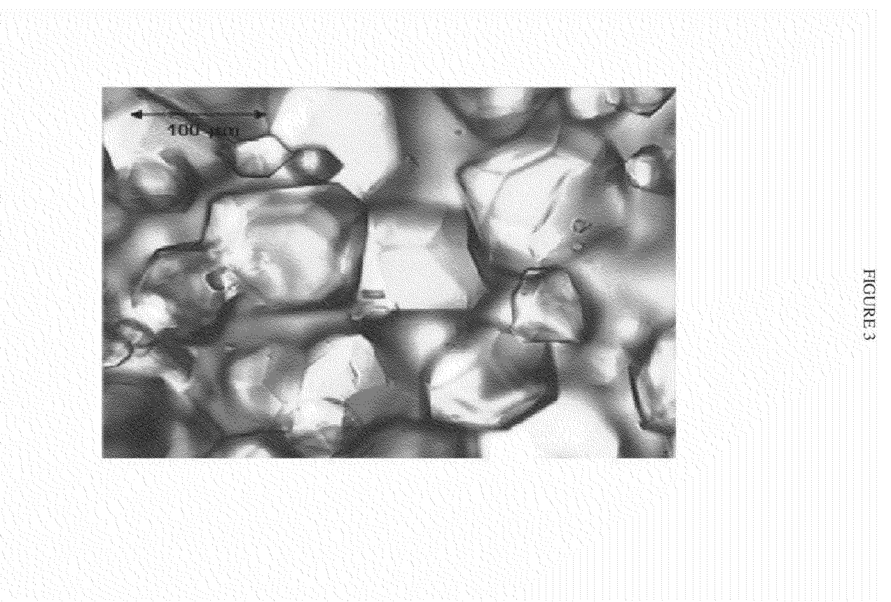

Linaclotide acetate (600 mg, 96% parity) was charged into a scintillation vial followed by a 96:4 mixture of ethylene glycol and water (3 mL). The suspension was then subjected to temperature cycling between 22° C. and 40° C. in 4 h cycles for a duration of 24 h. The slurry was then filtered by centrifugation. No acetate counterion was detected by ion chromatography. The XRPD and the PLM image of the material are shown respectively in FIGS. 1 and 3. Yield: quantitative.

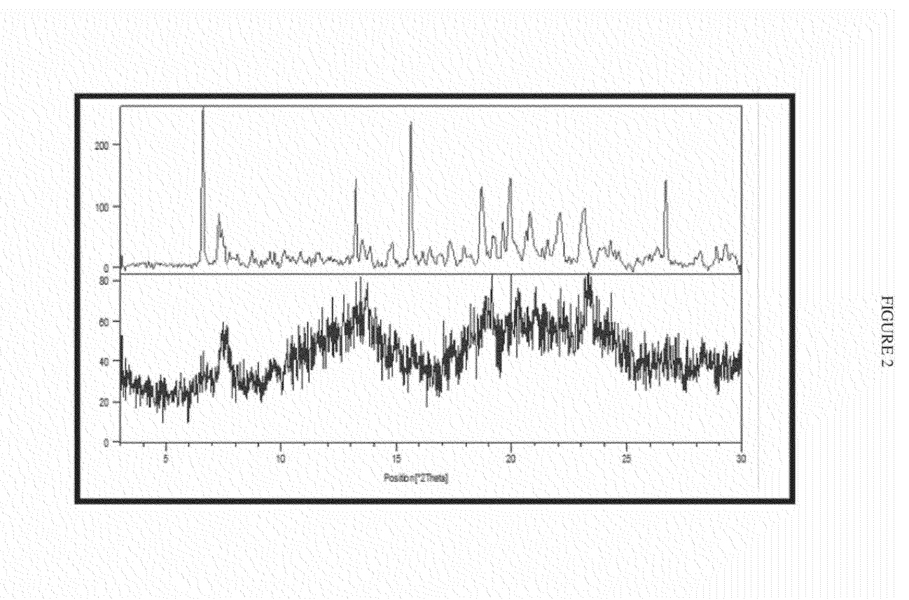

Linaclotide Form II was then washed with heptane and re-analysed by XRPD and HPLC. Amorphous Linaclotide with a purity of 98.70% was obtained in 90% yield. The XRPD comparison of amorphous vs. crystalline material is shown in FIG. 2.

### Example 2

Linaclotide acetate (600 mg, 96% purity) was charged into a scintillation vial followed by a 97:3 mixture of 1,3-propanediol and water (3 mL). The suspension was then subjected to temperature cycling between 22° C. and 40° C. in 4 h cycles for a duration of 24 h. The solid was then filtered by centrifugation and analysed by PLM and XRPD and found to be Linaclotide crystalline form II.

The resulting solid was then washed with heptane, re-analysed by XPRD and HPLC and found to be amorphous Linaclotide with a purity of 99%.

### Example 3

Linaclotide acetate (600 mg, 96% purity) was charged into a scintillation vial followed by NMP (3 mL). The suspension was then subjected to temperature cycling between 22° C. and 40° C. in 4 h cycles for a duration of 24 h. The solid was then filtered by centrifugation and analyzed by PLM and XRPD and found to be Linaclotide crystalline form II.

### Example 4

Linaclotide acetate (600 mg, 96% purity) was charged into a scintillation vial followed by DMSO (3 mL). The resulting clear solution was allowed to stand until all the solvent had evaporated. The solid was then analyzed by PLM and XRPD and found to be Linaclotide crystalline form II.

Other examples of embodiment of the invention include:
1. A crystalline form of Linaclotide characterised by a x-ray powder diffraction pattern comprising peaks at 2θ values of 6.6°, 15.6°, 18.7°, 19.9° and 23.1°, measured at a temperature of about 20° C. using Cu-Kα radiation (wavelength λ=1.5418 Å)
2. The crystalline form according to claim 1 further characterized by a x-ray powder diffraction pattern comprising 2θ values of 6.6°, 7.3°, 8.1°, 15.6°, 18.7°, 19.9°, 23.1° and 26.7°, measured at a temperature of about 20° C. and using Cu-Kα radiation (wavelength λ=1.5418 Å).
3. The crystalline form according to claims 1-2 in substantially pure form.
4. The crystalline form according to claims 1-3 including less than 10%, preferably less than 5%, more preferably less than 3%, most preferably less than 1% by weight of crystal form alpha, wherein form alpha is characterised by a X-ray powder diffraction pattern (XRPD) comprising peaks at 2θ values of 6.1°, 8.5°, 11.3°, 12.2° and 22.8°, measured at a temperature of about 20° C. and using Cu-Kα radiation (wavelength λ=5418 Å).
5. A pharmaceutical composition comprising the crystalline form of claim 1-4 and a pharmaceutically acceptable carrier of diluent.
6. The crystalline form of claims 1-4 or the composition of claim 5 for use for the treatment of gastrointestinal disorders and conditions, preferably irritable bowel syndrome and chronic constipation.
7. A process for the preparation of the crystalline form of claims 1-4 comprising the steps of suspending and/or stirring Linaclotide or Linaclotide acetate into a mixture comprising water and a suitable solvent to obtain a slurry and then isolating crystalline Linaclotide.
8. The process of claims 7, wherein the solvent is selected from the group consisting of diols and polar aprotic solvents.
9. The process of claims 7-8, wherein the solvent is selected from the group consisting of ethylene glycol, 1,2-propanediol, dimethyl sulfoxide (DMSO), N-methylpyrrolidone, dimethyl formamide and dimethyl acetamide.
10. The process of claims 7-9, wherein the water content in the solvent mixture is preferably below 20%, more preferably below 10%, even more preferably between 5% and 10%.
11. The process of claims 7-10, wherein the slurry is subjected to a temperature cycling regimen where each cycle, lasts 2 to 5 h, during each cycle the temperature starts at a first value, selected between 0° C. and 10° C., increases over time to a second value, selected between 17 and 27° C., and then drops back down to the first value, and each such cycle is repeated 5 to 25 times.
12. A process for the preparation of amorphous Linaclotide comprising preparing the crystalline form of claims 1-4 and converting it to amorphous Linaclotide by grinding, or by washing with a solvent selected from the group consisting of alcohols, ketones, ethers, hydrocarbons and water, or by drying in air or under vacuum.
13. Crystalline Linaclotide obtainable by the process of claims 7-11.

1. A crystalline solvate of Linaclotide having an orthorhombic P2₁2₁2₁ space group symmetry and the following unit cell dimensions: a=17.1+/−0.5 Å, b=21.8+/−0.7 Å, c=26.4+/−0.8 Å, α=90°, β=90°, γ=90° at −153° C.

2. The crystalline solvate of Linaclotide according to claim 1 further characterized by a x-ray powder diffraction pattern comprising peaks at 2θ values of 6.6°, 15.6°, 18.7°, 19.9° and 23.1°, measured at a temperature of about 20° C. and using Cu-Kα radiation (wavelength γ=1.5418 Å).

3. The crystalline solvate according to claim 1 further characterized by a x-ray powder diffraction pattern comprising 2θ values of 6.6°, 7.3°, 8.1°, 15.6°, 18.7°, 19.9°, 23.1° and 26.7°, measured at a temperature of about 20° C. and using Cu-Kα radiation (wavelength γ=1.5418 Å).

4. The crystalline solvate according to claim 1 characterized by a DSC trace showing a broad endotherm with onset at about 60° C. followed by two melting endotherms at 183° C. and 205° C.

5. The crystalline solvate according to claim 1 comprising a solvent selected from the group consisting of water, diols, polar aprotic solvents and mixtures thereof, preferably from the group consisting of water, ethylene glycol, 1,2-propanediol, 1,3-propanediol, dimethyl sulfoxide (DMSO), N-methylpyrrolidone (NMP), dimethyl formamide (DMF), dimethyl acetamide (DMA) and mixtures thereof, more preferably from the group consisting of water, ethylene glycol, 1,2-propanediol, 1,3-propanediol and mixtures thereof.

6. The crystalline solvate according to claim 1 in substantially pure form.

7. The crystalline solvate according to claim 1 including less than 10%, preferably less than 5%, more preferably less than 3%, most preferably less than 1% by weight of crystal form alpha, wherein form alpha is characterized by a x-ray powder diffraction pattern (XRPD) comprising peaks at 2θ values of 6.1°, 8.5°, 11.3°, 12.2° and 22.8°, measured at a temperature of about 20° C. and using Cu-Kα radiation (wavelength γ=1.5418 Å).

8. A pharmaceutical composition comprising the crystalline solvate of claim 1 and a pharmaceutically acceptable carrier or diluent.

9. The crystalline solvate of claim 1 or the composition of claim 8 for use for the treatment of gastrointestinal disorders and conditions, preferably irritable bowel syndrome and chronic constipation.

10. A process for the preparation of the crystalline solvate of claim 1 comprising the steps of suspending and/or stirring Linaclotide or Linaclotide acetate into a mixture comprising a solvent selected from the group consisting of water, diols, polar aprotic solvents and mixtures thereof to obtain a slurry and then isolating crystalline Linaclotide.

11. The process of claim 10, wherein the solvent is selected from the group consisting of water, ethylene glycol, 1,2-propanediol, 1,3-propanediol, dimethyl sulfoxide (DMSO), N-methylpyrrolidone (NMP), dimethyl formamide (DMF), dimethyl acetamide (DMA) and mixtures thereof, more preferably from the group consisting of water, ethylene glycol, 1,2-propanediol, 1,3-propanediol and mixtures thereof.

12. The process of claim 10, wherein the water content in the solvent mixture is preferably below 20%, more preferably below 10%, even more preferably between 5% and 10%.

13. The process of claim 10, wherein the slurry is subjected to a temperature cycling regimen where each cycle lasts 2 to 5 h, during each cycle the temperature starts at a first value, selected between 0° C. and 10° C., increases over time to a second value, selected between 17 and 27° C., and then drops back down to the first value, and each such cycle is repeated 5 to 25 times.

14. A process for the preparation of amorphous Linaclotide comprising preparing the crystalline form of claim 1 and converting it to amorphous Linaclotide by grinding, or by washing with a solvent selected from the group consisting of alcohols, ketones, ethers, hydrocarbons and water, or by drying in air or under vacuum.

15. Crystalline Linaclotide obtainable by the process of claim 10.
